(19) United States
(12) Patent Application Publication
(10) Pub. No.: US 2026/0157960 A1
(43) Pub. Date: Jun. 11, 2026

(54) RHEOLOGICALLY SYNOVIAL FLUID-LIKE HYDROGEL FORMULATIONS FOR USING IN INTRA-ARTICULAR APPLICATIONS

(71) Applicant: VSY BIYOTEKNOLOJI VE ILAC SANAYI ANONIM SIRKETI, Istanbul (TR)

(72) Inventors: Faruk OYTUN, Istanbul (TR); Efe CAN, Istanbul (TR); Busra Gizem KAYA, Istanbul (TR)

(73) Assignee: VSY BIYOTEKNOLOJI VE ILAC SANAYI ANONIM SIRKETI, Istanbul (TR)

(21) Appl. No.: 18/707,238

(22) PCT Filed: Nov. 3, 2021
(86) PCT No.: PCT/TR2021/051123

§ 371 Date: May 3, 2024

(51) Int. Cl. A61K 9/06; A61K 9/00; A61K 31/167; A61K 31/445; A61K 47/20; A61K 47/22; A61K 47/36; A61K 47/38

(52) U.S. Cl. CPC A61K 9/06; A61K 9/0019; A61K 31/167; A61K 31/445; A61K 47/20; A61K 47/22; A61K 47/36; A61K 47/38

(57) ABSTRACT

An injectable viscoelastic gel formulation suitable for use in intra-articular applications comprising at least one cellulose derivative, at least one sulfated glycosaminoglycan, at least one vitamin, at least one local anaesthetic and at least one antioxidant is provided.

### CROSS-REFERENCE TO RELATED APPLICATIONS

This application is the national phase entry of International Application No. PCT/TR2021/051123, filed on Nov. 3, 2021, the entire contents of which are incorporated herein by reference.

### TECHNICAL FIELD

The present invention relates to an injectable viscoelastic gel formulation suitable for use in intra-articular applications.

### BACKGROUND

Osteoarthritis (OA) is a degenerative disease affecting synovial joints and characterized by cartilage degradation, chronic inflammation, pain, stiffness and reduced mobility. OA has significant effects on the quality of life, especially by limiting many necessary daily activities such as walking or running. It is associated with increasing age because the articular cartilage of the joints may degrade with continual wear. An imbalance between the repair and degradation of the cartilage may disrupt the collagen matrix, resulting in OA. In OA, the functionalities are limited because the level of hyaluronic acid (HA) is lower (both molecular weight and concentration) than in healthy joints, decreasing the viscoelastic properties of the synovial fluid.

Synovial fluid is a joint lubricant that contains a high level of HA, which is physically beneficial by providing a protective barrier between the ends of the bones and improving lubrication due to its unique rheological properties, specifically its viscoelastic properties. It is usually present in a minimal amount in the synovial cavity, even in large joints, the average amount in humans is 0.13-3.5 mL. Typically, this fluid is pale yellow, clear, viscous, and contains soluble substances, including hyaluronic acid, growth factors, and cytokines. In OA the hyaluronic acid (HA) is depolymerized, causing deterioration in elastic and viscous properties of synovial fluid. In comparison to healthy synovial fluid, degenerated synovial fluid due to OA has lower viscosity. This reduction in lubrication properties of synovial fluid is due to decrease in molecular size and concentration of HA. Common therapies for OA often involve the use of analgesics, topical compounds, or the administration of solutions based on hyaluronic acid (HA) by infiltrative therapy. The most common non-surgical treatment includes the use of intra-articular hyaluronic acid (IA-HA) injection that is referred as viscosupplementation. The main aim of intra-articular injections of HA is to restore the mechanical properties of synovial joints and to reduce pain and stimulate specific biological responses that can improve the pathological condition. HA generally acts in the synovial fluid as a lubricant, protecting the articular surfaces from frictional damage, being responsible for supporting the viscoelasticity of biofluids and keeping synovial tissues from inflammatory mediators.

As an alternative to HA, the use of other polymers such as cellulose derivatives might provide several advantages mainly due to their similarity to the structure, composition and mechanical behavior of the components of the extracellular matrix (ECM). One such candidate polysaccharide is cellulose derivatives including hydroxypropyl cellulose (HPC), ethylcellulose (EC), methylcellulose (MC), hydroxyethylcellulose (HEC), hydroxypropylmethylcellulose (HPMC), and carboxymethylcellulose (CMC). They are water-soluble and the primary structural component of plant cell walls. Especially, CMC is a biocompatible, low-cost, FDA-approved material that is commercially available in high-purity forms, making this polymer a highly attractive option for biomedical applications.

Sulfated glycosaminoglycans (SGAGs) are linear polysaccharides consisting of repeating disaccharide units composed of N-acetylhexosamine and uronic acid, and exist as proteoglycans (PGs) by attaching to specific serine residues in the core protein. SGAGs are structurally classified into two groups, chondroitin sulfate/dermatan sulfate (CS/DS) and heparan sulfate/heparin (HS/Hep), on the basis of a difference in the repeating disaccharide unit. They are nutritional supplements that have recently gained widespread use as treatment options for OA. They potentially or theoretically act as chondroprotectors or/and as “disease-modifying OA drugs” offering not only symptomatic relief but also alteration of the natural history of OA. Slowing or even altering the inflammation and destructive effect on the articular cartilage and joint tissues. Chondroitin sulfate (CS) is a sulfated GAG being also a major component of the extracellular matrix of articular cartilage. It is found attached to proteins as part of the aggrecan of the cartilage. It plays a major role in creating considerable osmotic pressure that expands the matrix and places the collagen network under tension. It provides cartilage with resistance and elasticity allowing it to resist tensile stresses during various loading conditions. Studies have demonstrated that CS counteracts the action of IL-1b (a factor that induces articular inflammation and cartilage degeneration), thus playing a chondroprotective role. Additionally an effect on subchondral bone had been suggested by reducing the resorptive activity in subchondral bone. Proteoglycan content in cartilage was also significantly higher in animals treated with oral or intramuscular administration of CS than that in control animals. It has been shown that CS significantly decreases collagenolytic activity. Other studies suggested that the benefits of CS on degenerative osteoarthritic chondrocytes are larger than those on normal chondrocytes.

Osteoarthritis (OA) is a chronic degenerative joint disease, in which metabolic imbalance in bone is observed. The pathological mechanism of metabolic imbalance is not clear yet, but the nutritional factors, particularly the vitamins, might be intrinsic to the development and progression of OA. Vitamin D is a steroid hormone that plays an important role in the development and maintenance of the skeleton, as well as bone and cartilage metabolism. Vitamin D also has a role in bone mineralization, remodeling, bone formation, and immune homeostasis. The deficiency of vitamin D (<50 nmol/L) might be associated in the pathological process of OA by having a deleterious effect on calcium metabolism, osteoblastic activity, matrix ossification, bone remodeling, and bone density. There is evidence that vitamin D supplementation (i.e. with a higher level of 25 (OH)D or ergocalciferol) could protect against the development and worsening of OA [1]. Wang and colleagues reported that vitamin D supplementation significantly reduces metabolic-triggered inflammation in OA by decreasing serum levels of IL-6 and leptin-toadiponectin ratio and thus may have shown improvements in pain and physical function [2]. Pagès-Castelli and Prieto Alhambra suggested that vitamin D supplementation along with multidisciplinary interventions and bisphosphonates can prevent fractures among OA patients [3]. Vitamin E is a fat-soluble vitamin that exhibits an antioxidant activity. Vitamin E may enhance chondrocyte growth and exhibit an anti-inflammatory activity, as well as plays an important role in the prevention of cartilage degeneration [4]. The concentration of vitamin E in synovial fluid is reported to be down-regulated in OA patients, suggesting an imbalance between oxidative stress and antioxidant capacity. Oxidative stress is considered to play an important role in the progression of OA. The production of reactive oxygen species by activated chondrocytes can result in oxidative damage to various joint components including degradation of cartilage matrix and other specific proteins through the lipid peroxidation process. Vitamin K is a group of fat-soluble compounds, with two naturally occurring forms, vitamin K1 (phylloquinones) and vitamin K2 (menaquinones). In human OA cartilage, vitamin K deficiency produces abnormal growth plate calcification and inappropriate mineralization of cartilage. Thus, these fat-soluble vitamins play a key role in the pathophysiology of OA, and supplementation of these vitamins may provide innovative approaches for OA management [5]. Vitamin B family is made up of the most essential vitamins for the body. In several laboratory and clinical studies the analgesic and anti-inflammatory effects of them have been well illustrated. The antinociceptive effects of B vitamins for pain after spinal cord trauma have recently been shown by some researchers. The role of B vitamins deficiency in narrowing joint space and osteophytosis have been established, too. Based on these findings, it is likely that their use in OA, which is associated with inflammatory changes in joints, can be helpful, too. It seems that B vitamins can be used to reduce joint inflammation in OA, which may lead to less pain in patients. Interestingly, Muraki and colleagues [6] have found that low dietary intake of B1, B2, niacin and B6 are associated with narrowing of joint space in Japanese women.

Local anaesthetics, such as lidocaine and bupivacaine, are commonly used intra-articularly for therapeutic purposes and post-operatively in pain management for OA [7]. The intra-articular injection of local anesthetics (LAs), including lidocaine and bupivacaine, has commonly been used for the evaluation of a typical pain, as well as the temporary amelioration of pain in patients with osteoarthritis; it is generally accepted as safe [8]. It is known that amide local anaesthetics have potent and long lasting anti-inflammatory actions due to their structural similarity to steroid agents. The prototype, lidocaine, inhibits the release of inflammatory mediators such as histamine, leukotriene B4 and IL-1α and their functions like migration and adhesion. The potent anti-inflammatory effect of lidocaine on SPGN (sympathetic postganglionic neuron)-mediated plasma extravasations in the rat knee joint has been found to be due to direct inhibitory effects on synovial EPI receptors. The protective effects of lidocaine on inflamed articular surfaces might be attributed to its membrane stabilizing properties and the ability to change cellular functions. Thereby, intra-articular treatment with a sodium channel blocker like lidocaine might be considered to prevent arthritic pain-related behavior by interacting with cell membranes and stabilizing neuronal membrane effects, decreasing the inflammatory response and further inhibiting central nociceptive signaling.

Antioxidants are naturally occurring reducing agents capable of inhibiting ROS formation, scavenging free radicals and removing ROS derivatives. They are capable of inhibiting the oxidation of biological molecules. Antioxidants are thought to interfere with inflammatory reactions by being oxidized themselves. Antioxidant vitamins have major roles in modulating oxidative stress, regulating immune responses, and contributing to cell differentiation. Vitamin C (ascorbic acid), vitamin E, thiols (glutathione) and plant polyphenols have the capacity to neutralize ROS in joints and decrease the oxidative stress associated with the progression of arthritis.

U.S. Pat. No. 8,323,617 B2, known in the state of the art, discloses that HA formulations crosslinked with carbodiimide derivative crosslinking agents are used in the treatment of OA.

European patent document No. EP1443945 B1, known in the state of the art, discloses synergistic effect of sodium hyaluronate and chondroitin sulfate mixture on the lubrication and regeneration of articular cartilage damaged by stage I and stage II osteoarthritis of human joints. In this patent application, the combination of linear HA and chondroitin sulfate was used in the treatment of osteoarthritis.

In the U.S. Pat. No. 6,051,560 A, known in the state of the art, discloses chondroitin sulfate/sodium hyaluronate compositions, which are viscoelastic injections used in intraocular lens implantations, were prepared in a buffer solution, and it has been found that the strong interactions that occur between these structures exhibit increased solution stability and improved physical properties.

An article published by Falcone, S. J. et. al., known in the state of the art, discloses the use of hyaluronic acid in biomedical applications, its physical properties, rheological properties and differences of cross-linked HA from linear HA [9].

In the United States patent application No. US20130289131, known in the state of the art, the present invention relates to an injectable pharmaceutical formulation for the alleviation or reduction of joint irritation or for the reduction of worsening of existing joint inflammation, formulated for intra-articular injection comprising an active polyol ingredient, which polyol active ingredient is xylitol. Use of the intra-articular injectable formulations for the treatment of joint diseases or conditions including arthritis is described.

Although some beneficial effects in terms of reducing pain and improving joint functionality have been reported, at present the remission of the pathology remains a challenge. In spite of the common use of HA-based solutions for OA treatment, these products display several disadvantages such as the fast degradation of HA molecules when injected into the joints. The degradation of HA is mainly due to the action of hyaluronidases and reactive oxygen species (ROS). This aspect limits the permanence of HA at the intra-articular level and accounts for the repeated number of injections that must be performed in a short time frame. Furthermore, the major disadvantage of this type of injection is the invasion of the joint space, enhancing the possibility of subsequent joint infection and recurrent administrations.

### SUMMARY

The objective of the present invention is to provide an injectable viscoelastic gel formulation suitable for use in intra-articular applications.

Another objective of the present invention is to eliminate several disadvantages of the applications used in the technical field such as the fast degradation of HA molecules when injected into the joints.

Another objective of the present invention is to obtain similar rheological properties of synovial fluid.

In an embodiment of the invention an injectable viscoelastic gel formulation for intra-articular use contains a cellulose derivative, a sulfated glycosaminoglycan, a vitamin, and an antioxidant.

In a preferred embodiment of the invention, the injectable viscoelastic gel formulation for intra-articular use further includes a local anesthetic.

In a preferred embodiment of the invention the cellulose derivative may be hydroxypropyl cellulose (HPC), ethylcellulose (EC), methylcellulose (MC), hydroxyethylcellulose (HEC), hydroxypropylmethylcellulose (HPMC), and carboxymethylcellulose (CMC) or combinations thereof.

In a preferred embodiment of the invention the sulfated glycosaminoglycan (SGAG) may be chondroitin sulfate (CS), dermatan sulfate (DS) and heparan sulfate (HS) and heparin (Hep) or combinations thereof.

In a preferred embodiment of the invention the vitamin may be vitamin A derivatives such as retinal, retinol, pro-vitamin A, retinoic acid, vitamin B derivatives such as vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), vitamin C derivatives L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate, vitamin D and vitamin K derivatives or combinations thereof.

In a further preferred embodiment of the invention the injectable viscoelastic gel formulation for intra-articular use further includes a local anesthetic, and the local anesthetic may be lidocaine hydrochloride, bupivacaine hydrochloride, mepivacaine hydrochloride, levobupivacaine hydrochloride and ropivacaine hydrochloride or combinations thereof.

In a preferred embodiment of the invention, the antioxidant may be glutathione, allicin, astaxanthin, N-Acetylcarnosine (NAC), epigallocatechin gallate (EGCG), coenzyme Q10 (CoQ10), quercetin, alpha lipoic acid, resveratrol, alpha tocopherol, carotene, beta carotene, trolox, hydroxytyrosol, tyrosol, ferulic acid, caffeic acid, rutin, diosmin, melatonin, taurine and hypotaurine or combinations thereof.

### BRIEF DESCRIPTION OF THE DRAWINGS

In this detailed description, the preferred embodiments of the injectable viscoelastic gel according to the invention are only disclosed for better understanding of the subject without forming any limiting effect.

The FIGURE. Plotting about the log of the zero shear viscosity (η₀) versus the log of the shear rate (Hz) for CMC samples.

### DETAILED DESCRIPTION OF THE EMBODIMENTS

The invention relates to an injectable viscoelastic gel formulation suitable for use in intra-articular applications. The ingredients of the viscoelastic gel formulation are prepared in phosphate buffer solution. This formulation comprises at least one cellulose derivative including, but not limited to, those selected from hydroxypropyl cellulose (HPC), ethylcellulose (EC), methylcellulose (MC), hydroxyethylcellulose (HEC), hydroxypropylmethylcellulose (HPMC), and carboxymethylcellulose (CMC) or combinations thereof. Cellulose derivatives provide the needed lubrication for the viscoelastic gel formulation and have an effect of the prevention of collagen destruction and take part in tissue repair and wound healing. They adapt to changing shear forces with dynamic viscosity and act as a shock absorber, exhibit elastic and suspensive behavior. In a preferred embodiment of the invention, the cellulose derivative is carboxymethylcellulose (CMC). The amount of the cellulose derivatives in the formulation is between 0.5-5% by weight based on the total weight of the formulation.

The viscoelastic gel formulation according to the invention comprises at least one sulfated glycosaminoglycan (SGAG) including, but not limited to, those selected from chondroitin sulfate (CS), dermatan sulfate (DS) and heparan sulfate (HS) and heparin (Hep) or combinations thereof. In a preferred embodiment of the invention, the sulfated glycosaminoglycan (SGAG) is chondroitin sulfate. The amount of the sulfated glycosaminoglycan (SGAG) in the formulation is between 0.5-5% by weight based on the total weight of the formulation. The technical effects provided due to the fact that the viscoelastic gel formulation comprises sulfated glycosaminoglycans (SGAGs) are listed below:
SGAGs provide cartilage with resistance and elasticity allowing it to resist tensile stresses during various loading conditions.
Due to the negative charge of SGAGs, they are responsible for the water retention of the cartilage, which is important for pressure resistance.
SGAGs have an excellent safety profile, therefore there are no safety concerns for long-term use.
SGAGs increases the hyaluronan production by human synovial cells, which has a beneficial effect on maintaining viscosity in the synovial fluid.
SGAGs stimulate the chondrocyte metabolism, leading to the synthesis of collagen and proteoglycan, the basic components of new cartilage.
SGAGs inhibit the enzymes leukocyte elastase and hyaluronidase, which are found in high concentration in the synovial fluid of patients with rheumatic diseases.

The viscoelastic gel formulation according to the invention comprises at least one vitamin including, but not limited to, those selected from vitamin A derivatives such as retinal, retinol, pro-vitamin A, retinoic acid, vitamin B derivatives such as vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), vitamin C derivatives L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate, vitamin D and vitamin K derivatives or combinations thereof. In a preferred embodiment of the invention, the vitamin is Vitamin B3. The amount of the vitamin in the formulation is between 0.01-3% by weight based on the total weight of the formulation. Vitamins in viscoelastic gel formulation play a role in increasing the collagen synthesis and soft tissue healing. They neutralize the reactive oxygen species (ROS) caused by inflammation. Vitamins can be used to reduce joint inflammation in OA, which may lead to less pain in patients. In addition, they may enhance chondrocyte growth and exhibit an anti-inflammatory activity, as well as plays an important role in the prevention of cartilage degeneration.

The viscoelastic gel formulation according to the invention comprises at least one local anaesthetic including, but not limited to, those selected from lidocaine hydrochloride, bupivacaine hydrochloride, mepivacaine hydrochloride, levobupivacaine hydrochloride and ropivacaine hydrochloride or combinations thereof. In a preferred embodiment of the invention, the local anaesthetic is lidocaine hydrochloride. The amount of the local anaesthetics in the formulation is between 0.01-0.5% by weight based on the total weight of the formulation. The local anaesthetics is known to have potent and long lasting anti-inflammatory actions due to their structural similarity to steroid agents. They inhibit the release of inflammatory mediators such as histamine, leukotriene B4 and IL-1α and their functions like migration and adhesion.

The viscoelastic gel formulation according to the invention comprises at least one antioxidant including, but not limited to, those selected from glutathione, allicin, astaxanthin, N-Acetylcarnosine (NAC), epigallocatechin gallate (EGCG), coenzyme Q10 (CoQ10), quercetin, alpha lipoic acid, resveratrol, alpha tocopherol, carotene, beta carotene, trolox, hydroxytyrosol, tyrosol, ferulic acid, caffeic acid, rutin, diosmin, melatonin, taurine and hypotaurine or combinations thereof. In a preferred embodiment of the invention, the antioxidant is glutathione. The amount of the antioxidant in the formulation is between 0.01-3% by weight based on the total weight of the formulation. Antioxidants are important in terms of significantly reducing chronic mild to moderate knee joint pain. They effectively suppress the expression levels of matrix metalloproteinases (MMPs) at the transcriptional level in inflamed knee joint tissues. They reduce cartilage degradation by maintaining cartilage homeostasis over Nrf2 in osteoarthritis. They also have an effect on inhibiting the PI3K/Akt/NF-κB pathway in chondrocytes and inhibiting the expression of IL-1β-induced inflammatory mediators, exerting a therapeutic effect in osteoarthritis and reducing cartilage destruction.

An example of the formulation of the invention is explained below. Example 1 is given only to demonstrate a preferred application of the invention and to prove the effectiveness of the invention, without any limiting effect.

### Example 1

### Preparation of Hydrogels Based on Cellulose Derivatives

10 wt % of carboxymethyl cellulose (CMC) was first dissolved in 0.25 M sodium hydroxide (NaOH) solution by a mechanical stirrer. After the CMC was completely dissolved, BDDE (1 wt % with respect to the total mixture) was added to the solution and stirred for 15 min. The cross-link reaction was then performed at 40° C. for 2 hours. After the reaction, the hydrogels were put into phosphate buffer solution (PBS) following by neutralization with 0.1 M of hydrochloric acid (HCl). The hydrogels were then repeatedly washed and swollen in PBS to remove unreacted substances.

The resulting hydrogels at different gel:fluid ratio were prepared according to the amounts listed in the following Table 1 using cross-linked CMC, non-cross-linked CMC, chondroitin sulfate selected from the SGAG group, Vitamin B3 selected from the Vitamin group, lidocaine hydrochloride selected from the local anesthetic group and glutathione selected from the antioxidant group. Finally, the hydrogels were filled into the syringes and sterilized in air-stream at 121° C. for 15 min. Gel:fluid ratio represents the ratio of crosslinked CMC (gel) to non-crosslinked CMC (fluid).

TABLE 1

| | CMC-1 | CMC-2 | CMC-3 | CMC-4 |
|---|---|---|---|---|
| | Gel:fluid | Gel:fluid | Gel:fluid | Gel:fluid |
| | ratio | ratio | ratio | ratio |
| | 50:50 | 40:60 | 30:70 | 20:80 |
| Crosslinked CMC | 12 | 9.6 | 7.2 | 4.8 |
| (mg/ml) | | | | |
| Non-Crosslinked CMC | 12 | 14.4 | 16.8 | 19.2 |
| (mg/ml) | | | | |
| Chondroitin Sulfate | 30 | 30 | 30 | 30 |
| (mg/ml) | | | | |
| Lidocaine HCl (mg/ml) | 3 | 3 | 3 | 3 |
| Vitamin B3 (mg/ml) | 10 | 10 | 10 | 10 |
| Glutathione (mg/ml) | 5 | 5 | 5 | 5 |

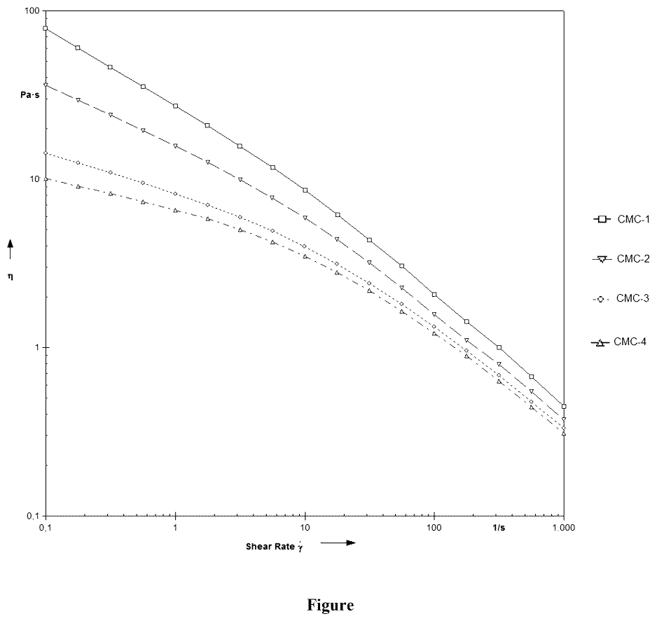

Zero shear rate viscosity of healthy knee synovial fluid has been studied by many research groups in the literature. Fam and colleagues [10] observed that zero shear viscosity of synovial fluid in healthy joints ranges from 1 to 175 Pa·s. The creep behavior for CMC hydrogels at various gel:fluid ratios were measured; the log of the zero-shear viscosity, π₀, plotted versus the log of the shear rate (Hz) is shown in FIG. 1. All CMC samples exhibited non-Newtonian shear thinning behavior; that is viscosity decreases with increasing shear rate (FIG. 1). This is the characteristic for healthy SF. The highest viscosity was measured for CMC-1 with a 50:50 gel:fluid ratio and the lowest for CMC-4 with a 20:80 gel:fluid ratio over the whole range of shear rate. All formulations had zero shear rate viscosities that inside of the range for zero shear viscosity of healthy knee joints as shown in Table 2. The data indicates that the viscosity is strongly dependent on the gel:fluid ratios. Results showed that rheological behaviors of CMC samples varied widely. The variability is demonstrated by the wide range of zero shear viscosity. The zero shear viscosity (π₀) varied from 10.1 to 78.5 Pa·s (Table 2).

TABLE 2
Zero shear viscosity of hydrogel samples

| | Zero Shear |
|---|---|
| | Viscosity |
| Sample | (Pa · s) |
| CMC-1 | 78.5 |
| CMC-2 | 35 |
| CMC-3 | 14.2 |
| CMC-4 | 10.1 |

### REFERENCES

[1]. Zheng, X. Y., Liang, J., Li, Y. S., & Tu, M. (2018). Role of fat-soluble vitamins in osteoarthritis management. JCR: Journal of Clinical Rheumatology, 24(3), 132-137.
[2]. Wang, X., Hunter, D., Xu, J., & Ding, C. (2015). Metabolic triggered inflammation in osteoarthritis. Osteoarthritis and cartilage, 23(1), 22-30.
[3]. Prieto Alhambra, D. (2013). Degenerative osteoarthritis, osteoporosis and fractures: controversies and evidences. Medicina clinica, 141(5).
[4]. Brand, C., Snaddon, J., Bailey, M., & Cicuttini, F. (2001). Vitamin E is ineffective for symptomatic relief of knee osteoarthritis: a six month double blind, randomised, placebo controlled study. Annals of the rheumatic diseases, 60(10), 946-949.
[5]. Wallin, R., Schurgers, L. J., & Loeser, R. F. (2010). Biosynthesis of the vitamin K-dependent matrix Gla protein (MGP) in chondrocytes: a fetuin-MGP protein complex is assembled in vesicles shed from normal but not from osteoarthritic chondrocytes. Osteoarthritis and cartilage, 18(8), 1096-1103.
[6]. Muraki, Shigeyuki, et al. “Association of dietary intake with joint space narrowing and osteophytosis at the knee in Japanese men and women: the ROAD study.” Modern rheumatology 24.2 (2014): 236-242.
[7]. Onur, T. S., Sitron, C. S., & Dang, A. (2013). Co-administration of hyaluronic acid with local anaesthetics shows lower cytotoxicity than local anaesthetic treatment alone in bovine articular chondrocytes. Bone & joint research, 2(12), 270-275.
[8]. Lee, Y. J., Kim, S. A, & Lee, S. H. (2016). Hyaluronan suppresses lidocaine-induced apoptosis of human chondrocytes in vitro by inhibiting the p53-dependent mitochondrial apoptotic pathway. Acta Pharmacologica Sinica, 37(5), 664-673.
[9]. Falcone, S. J., Palmeri, D., & Berg, R. A. (2006). Biomedical applications of hyaluronic acid.
[10]. Farn, H., Bryant, J. T., & Kontopoulou, M. (2007). Rheological properties of synovial fluids. Biorheology, 44(2), 59-74.

What is claimed is:

1. An injectable viscoelastic gel formulation suitable for intra-articular use in, the viscoelastic gel formulation comprising:
at least one cellulose derivative,
at least one sulfated glycosaminoglycan(SGAG),
at least one vitamin, and
at least one antioxidant.

2. The injectable viscoelastic gel formulation according to claim 1, further comprising at least one local anesthetic.

3. The injectable viscoelastic gel formulation according to claim 1, wherein the at least one cellulose derivative comprises hydroxypropyl cellulose (HPC), ethylcellulose (EC), methylcellulose (MC), hydroxyethylcellulose (HEC), hydroxypropylmethylcellulose (HPMC), carboxymethylcellulose (CMC), or combinations thereof.

4. The injectable viscoelastic gel formulation according to claim 1, wherein the at least one sulfated glycosaminoglycan (SGAG) comprises chondroitin sulfate (CS), dermatan sulfate (DS), heparan sulfate (HS), heparin (Hep), or combinations thereof.

5. The injectable viscoelastic gel formulation according to claim 1, wherein the at least one vitamin comprises vitamin A, retinal, retinol, pro-vitamin A, retinoic acid, vitamin B, vitamin B1 (thiamine), vitamin B2 (riboflavin), vitamin B3 (nicotinamide), vitamin B5 (pantothenic acid), vitamin B6 (pyridoxine), vitamin B8 (biotin), vitamin B9 (folacin), vitamin B12 (cobalamins), vitamin C, L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate, vitamin D, vitamin K, or combinations thereof.

6. The injectable viscoelastic gel formulation according to claim 2, wherein the at least one local anesthetic comprises lidocaine hydrochloride, bupivacaine hydrochloride, mepivacaine hydrochloride, levobupivacaine hydrochloride, ropivacaine hydrochloride, or combinations thereof.

7. The injectable viscoelastic gel formulation according to claim 1, wherein the at least one antioxidant comprises glutathione, allicin, astaxanthin, N-Acetylcarnosine (NAC), epigallocatechin gallate (EGCG), coenzyme Q10 (CoQ10), quercetin, alpha lipoic acid, resveratrol, alpha tocopherol, carotene, beta carotene, trolox, hydroxytyrosol, tyrosol, ferulic acid, caffeic acid, rutin, diosmin, melatonin, taurine, hypotaurine, or combinations thereof.
